(19) United States
(12) Patent Application Publication
(10) Pub. No.: US 2009/0118301 A1
(43) Pub. Date: May 7, 2009

(54) Compositions and Methods for Treating Cancer

(75) Inventors: Peter S. Lu, Palo Alto, CA (US); Michael P. Belmares, San Jose, CA (US); Jingjing Wang, Sunnyvale, CA (US); Johannes Schweizer, Mountain View, CA (US); Aida A. Fantaye, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation, Sunnyvale, CA (US)

(21) Appl. No.: 12/262,023

(22) Filed: Oct. 30, 2008

Related U.S. Application Data
(60) Provisional application No. 60/985,196, filed on Nov. 2, 2007.

(51) Int. Cl. A61K 31/497; A61K 31/40; A61K 31/34; C07D 403/14; C07D 403/04; A61K 31/4164

(52) U.S. Cl. 514/254.09; 514/414; 514/412; 514/395; 514/469; 548/455; 544/373

(57) ABSTRACT

The invention provides compounds useful for treating or effecting prophylaxis of cancers, particularly cervical cancer.

### CROSS-REFERENCE TO RELATED APPLICATION

This application claims the benefit of U.S. Provisional App. No. 60/985,196 filed Nov. 2, 2007, incorporated by reference in its entirety.

### BACKGROUND OF THE INVENTION

Cervical cancer is the second most common cancer diagnosis in women and is linked to high-risk human papillomavirus infection 99.7% of the time. In the year 2007, for example, the National Cancer Institute reports that an estimated 3,670 deaths will be ascribed to this type of cancer and approximately 11,150 new cases will be diagnosed in US women. Furthermore, there are approximately 400,000 cases of cervical cancer and close to 200,000 deaths annually worldwide. Human papilloma viruses (HPVs) are one of the most common causes of sexually transmitted disease in the world. Overall, 50-75% of sexually active men and women acquire genital HPV infections at some point in their lives. An estimated 5.5 million people become infected with HPV each year in the US alone, and at least 20 million are currently infected. The more than 100 different isolates of HPV have been broadly subdivided into high-risk and low-risk subtypes based on their association with cervical carcinomas or with benign cervical lesions or dysplasias.

A number of lines of evidence point to HPV infections as the etiological agents of cervical cancers. Multiple studies in the 1980's reported the presence of HPV variants in cervical dysplasias, cancer, and in cell lines derived from cervical cancer. Further research demonstrated that the E6-E7 region of the genome from oncogenic HPV 18 is selectively retained in cervical cancer cells, suggesting that HPV infection could be causative and that continued expression of the E6-E7 region is required for maintenance of the immortalized or cancerous state. The following year, Sedman et al. demonstrated that the E6-E7 genes from HPV 16 were sufficient to immortalize human keratinocytes in culture. Barbosa et al. demonstrated that although E6-E7 genes from high risk HPVs could transform cell lines, the E6-E7 regions from low risk, or non-oncogenic variants such as HPV 6 and HPV 11 were unable to transform human keratinocytes. More recently, Pillai et al. examined HPV 16 and 18 infection by in situ hybridization and E6 protein expression by immunocytochemistry in 623 cervical tissue samples at various stages of tumor progression and found a significant correlation between histological abnormality and HPV infection.

Human papillomaviruses characterized to date are associated with lesions confined to the epithelial layers of skin, or oral, pharyngeal, respiratory, and, most importantly, anogenital mucosae. Specific human papillomavirus types, including HPV 6 and 11, frequently cause benign mucosal lesions, whereas other types such as HPV 16, 18, and a host of other strains, are predominantly found in high-grade lesions and cancer. Individual types of human papillomaviruses (HPV) which infect mucosal surfaces have been implicated as the causative agents for carcinomas of the cervix, anus, penis, larynx and the buccal cavity, occasional periungal carcinomas, as well as benign anogenital warts. The identification of particular HPV types is used for identifying patients with premalignant lesions who are at risk of progression to malignancy. Although visible anogenital lesions are present in some persons infected with human papillomavirus, the majority of individuals with HPV genital tract infection do not have clinically apparent disease, but analysis of cytomorphological traits present in cervical smears can be used to detect HPV infection. Papanicolaou tests are a valuable screening tool, but they miss a large proportion of HPV-infected persons due to the unfortunate false positive and false negative test results. In addition, they are not amenable to worldwide testing because interpretation of results requires trained pathologists. Because of the limited use and success rate of the Papanicolaou test, many HPV-infected individuals fail to receive timely diagnosis, a problem that precludes efforts to administer treatment prior to the appearance of clinical symptoms. A significant unmet need exists for early and accurate diagnosis of oncogenic HPV infection as well as for treatments directed at the causative HPV infection, preventing the development of cervical cancer by intervening earlier in disease progression.

Because treatments are usually administered after the onset of clinical symptoms, current treatment paradigms are focused on the actual cervical dysplasia rather than the underlying infection with HPV. Women are screened by physicians annually for cervical dysplasia and are treated with superficial ablative techniques, including cryosurgery, laser ablation and excision. As the disease progresses, treatment options become more aggressive, including partial or radical hysterectomy, radiation or chemotherapy. All of these treatments are invasive and carry the possibility or guarantee of permanent infertility. In addition, surgical removal of tissue may not guarantee that all infected cells have been eliminated due to the fact that some transformed cells may not yet be displaying the morphological changes associated with HPV infection.

### SUMMARY OF THE CLAIMED INVENTION

The invention provides compounds conforming to formulae described below. Such compounds preferably inhibits binding of binding of HPV E6 protein to a polypeptide comprising the amino acid sequence of a first PDZ domain from MAGI-1.

The invention further provides a pharmaceutical composition comprising such a compound. Such compositions are preferably manufactured under GMP conditions. The compounds of the invention are preferably provided in at least 99% pure form.

The invention further provides a method of treating or effecting prophylaxis against an infection by an oncogenic human papilloma virus, comprising administering to a subject having or at risk of HPV infection an effective regime of a compound of any preceding claim, whereby the compound treats or effects prophylaxis of the infection or its sequellae.

In some methods, the subject is infected with HPV. In some methods, the subject has cervical cancer. In some methods, the subject has cervical dysplasia. In some methods, the subject is at risk of HPV infection.

The invention further provides methods of treating or effecting prophylaxis of cancer. Such methods entail administering to a subject having or at risk of cancer an effective regime of a compound of any of claims 1-14, whereby the compound treats or effects prophylaxis of cancer. In some such methods, the subject is infected with an oncogenic human papilloma virus. In some such methods, the cancer is cervical cancer, vaginal cancer, anal cancer or head and neck cancer. In some such methods, the cancer is breast cancer, ovarian cancer, brain cancer, leukemia or lymphoma.

The invention further provides methods of treating cervical cancer. Such methods entail administering to a subject having cervical cancer an effective regime of any of the compounds described herein, whereby the compound treats the cervical cancer.

### BRIEF DESCRIPTION OF THE DRAWINGS

FIG. 1 shows the effect of compound 7291-0042 on cell migration.

FIG. 2A shows the effect of compound 7291-0042 on cell proliferation for 3Y1 pBlast E6-16 (wild type) cells.

FIG. 2B shows the effect of compound 7291-0042 on cell proliferation for 3Y1 pBlast E6-16 ΔPL cells.

FIG. 2C shows the effect of compound 7291-0042 on cell proliferation for 3Y1 pBlast cells.

FIG. 3 shows the cytotoxic effect of compound 7291-0042 for E6 cervical cancer cells, C33a, HeLa and SiHa.

FIGS. 4A and 4B shows E6 protein expression in HeLa cells treated with compound 7291-0042 and without.

FIG. 5 shows p53 tumor suppressor protein expression in HeLa cells treated with compound 7291-0042 and without.

FIG. 6A shows other dimers of the 7291-0042-type drug.

FIG. 6B shows variants of the 7291-0042 drug with COOH mimics or headgroups. If the main scaffold is kept unchanged many head groups can be used including —O—CH2-COO— in 7291-0042.

FIG. 7 shows that 7291-0042 selectively induces apoptosis in HPV-positive cervical cancer cells, as measured by a TUNEL assay in HeLa and C33A cells after treatment with 7291-0042 for 48 hours.

### DEFINITIONS

The term “human papillomavirus” or “HPV” refers to a diverse group of DNA-based viruses that are one of the most common causes of sexually transmitted disease in the world. Cervical cancer is identified to be caused by HPV. The more than 100 different isolates of HPV have been broadly subdivided into high-risk and low-risk subtypes based on their association with cervical carcinomas or with benign cervical lesions or dysplasias. These HPV isolates are sometimes referred to as HPV strains or types and are often designated or referred to by number only or by “HPV #”, where “#” is the number of the oncogenic or cancer causing genotype.

An “oncogenic HPV strain” is an HPV strain that is known to cause cervical cancer as determined by the National Cancer Institute (NCI, 2001). Exemplary oncogenic strains are HPV16, HPV33, HPV35, HPV52, HPV58 and HPV66. Oncogenic strains of HPV not specifically listed here can be found at the world wide website of the National Center for Biotechnology Information (NCBI).

“Oncogenic E6 proteins,” “E6” or “E6 oncoprotein” used interchangeably are E6 proteins encoded by the above oncogenic HPV strains such as the high-risk HPV types 16 and 18 (see, e.g., NCBI Taxonomy IDs: 333760 and 333761, respectively). E6 is a protein for viral replication as well as for host cell immortalization and transformation. E6 binds to proteins with PDZ-domains such as the MAGUK (membrane-associated guanylate kinase family) proteins (described below). These proteins include MAGI-1, MAGI-2, and MAGI-3. When E6 complexes with the PDZ domains on the MAGI proteins, the complex distorts DLG proteins' shape and thereby impedes their function. E6 also binds p53, a tumor suppressor protein that negatively regulates cell cycle progression, cell growth and division. Binding of E6 to p53 results in the ubiquination and eventual degradation of the p53 protein, which process involves another cellular protein termed “E6-associated protein”. Consequently, cells expressing E6 will have a reduced basal level of p53.

The term “PDZ domain” refers to protein sequence (i.e., modular protein domain) of less than approximately 90 amino acids, (i.e., about 80-90, about 70-80, about 60-70 or about 50-60 amino acids), characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats. PDZ domains generally appear to maintain a core consensus sequence (Doyle 1996, Cell 85: 1067-76).

PDZ domains are found in diverse membrane-associated proteins including members of the MAGUK family of guanylate kinase homologs, several protein phosphatases and kinases, neuronal nitric oxide synthase, tumor suppressor proteins, and several dystrophin-associated proteins, collectively known as syntrophins.

The term “PDZ domain” also encompasses variants (e.g., naturally occurring variants) of the sequences (e.g., polymorphic variants, variants with conservative substitutions, and the like) and domains from alternative species (e.g. mouse, rat). Typically, PDZ domains are substantially identical to those shown in U.S. patent application Ser. No. 09/724,553, e.g., at least about 70%, at least about 80%, or at least about 90% amino acid residue identity when compared and aligned for maximum correspondence. PDZ domains can be mutated to give amino acid changes that can strengthen or weaken binding and to alter specificity, yet they remain PDZ domains (Schneider et al, 1998, Nat. Biotech. 17:170-5). Unless otherwise indicated, a reference to a particular PDZ domain (e.g., a MAGI-1 domain 2) is intended to encompass the particular PDZ domain and HPV E6-binding variants thereof In other words, if a reference is made to a particular PDZ domain, a reference is also made to variants of that PDZ domain that bind an oncogenic E6 protein of HPV, as described below. In this respect it is noted that the numbering of PDZ domains in a protein can change.

The term “PDZ protein” refers to a naturally occurring protein containing a PDZ domain. Exemplary PDZ proteins include CASK, MPP1, DLG1, DLG2, PSD95, NeDLG, TIP-33, SYN1a, TIP-43, LDP, LIM, LIMK1, LIMK2, MPP2, NOS1, AF6, PTN-4, prIL16, 41.8 kD, KIAA0559, RGS12, KIAA0316, DVL1, TIP-40, TIAM1, MINT1, MAGI-1, MAGI-2, MAGI-3, KIAA0303, CBP, MINT3, TIP-2, KIAA0561, and TIP-1.

The term “PDZ-domain polypeptide” refers to a polypeptide containing a PDZ domain, such as a fusion protein including a PDZ domain sequence, a naturally occurring PDZ protein, or an isolated PDZ domain peptide. A PDZ-domain polypeptide can therefore be about 60 amino acids or more in length, about 70 amino acids or more in length, about 80 amino acids or more in length, about 90 amino acids or more in length, about 100 amino acids or more in length, about 200 amino acids or more in length, about 300 amino acids or more in length, about 500 amino acids or more in length, about 800 amino acids or more in length, about 1000 amino acids or more in length, usually up to about 2000 amino acids or more in length. PDZ domain peptides are usually no more than about 100 amino acids (e.g., 50-60 amino acids, 60-70 amino acids, 80-90 amino acids, or 90-100 amino acids), and encode a PDZ domain.

The term “PL protein” or “PDZ Ligand protein” refers to a polypeptide that can be a naturally-occurring or non-naturally occurring peptide, that forms a molecular complex with a PDZ-domain, or to a protein whose carboxyl-terminus, when expressed separately from the full length protein (e.g., as a peptide of 4-25 residues, e.g., 8, 10, 12, 14 or 16 residues), forms such a molecular complex.

“MAGI-1”, “membrane-associated guanylate kinase inverted 1”, MAGi1 or “membrane associated guanylate kinase, WW and PDZ domain containing 1” is a member of the membrane-associated guanylate kinase homologue (MAGUK) family. MAGUK proteins participate in the assembly of multiprotein complexes on the inner surface of the plasma membrane at regions of cell-cell contact. The product of this gene can play a role as scaffolding protein at cell-cell junctions. Alternatively spliced transcript variants encoding different isoforms have been identified. MAGI-1 is also known as atrophin-1-interacting protein 3 (AIP3); BAI1-associated protein 1 (BAP1 or BAIAP1); WW domain-containing protein 3 (WWP3); Trinucleotide repeat-containing gene 19 protein (TNRC19). See Shiratsuchi T, et al., (1998) Biochem. Biophys. Res. Commun. 247:597-604.

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

Compounds are typically substantially pure from undesired contaminant. This means that an agent is typically at least about 50% w/w (weight/weight) purity, as well as being substantially free from interfering contaminants, such unreacted reagents or byproducts in their synthesis. Sometimes the compounds are at least about 80% w/w and, more preferably at least 90 or about 95% or 99% w/w purity.

The term subject includes humans, animals subject to cancers, particularly domestic animals and laboratory animals.

### DETAILED DESCRIPTION OF THE INVENTION

### I. General

Human papillomaviruses (HPV) associated with oncogenesis differ from HPV strains not associated with oncogenesis in that the E6 protein of HPV strains associated with oncogenesis has a PL region that interacts with cellular PDZ proteins including MAGI-1. The inventors have found compounds that inhibit such interaction. The compounds are useful for treating or effecting prophylaxis of HPV infection including its sequellae, particularly cervical dysplasia and cervical cancer. The compounds also enhance expression of the tumor suppressor P53 irrespective of HPV infection and are thus useful for treatment and prophylaxis of other types of cancer as well.

### II. Compounds of the Invention

The invention provides compounds of formula I.

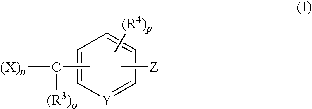

Each X is a heteroaryl ring system having from 5 to 10 ring atoms wherein from 1 to 4 ring atoms are heteroatoms each independently selected from the group consisting of N, O and S, wherein the heteroaryl ring system is substituted with from 0 to 6 R¹ groups;

Each R¹ is independently selected from the group consisting of H, C₁₋₆ alkyl, C₂₋₆ alkenyl, C₂₋₆ alkynyl, C₁₋₆ alkoxy, halogen, C₁₋₆ haloalkyl, —C₀₋₆ alkyl—OR^{1a}, —NR^{1a}R^{1b}, —CN, —C(O)R^{1a}, —C(O)OR^{1a}, —OC(O)R^{1a}, —C(O)NR^{1a}R^{1b}, —N(R^{1a})C(O)R^{1b}, —OC(O)NR^{1a}R^{1b}, —N(R^{1a})C(O)OR^{1b}, —NR^{1a}C(O)NR^{1b}R^{1c}, —NO₂, —C₀₋₆ alkyl-aryl, heteroaryl, cycloalkyl and heterocycloalkyl;

Each of R^{1a}, R^{1b} and R^{1c} is independently selected from the group consisting of H and C₁₋₆ alkyl;

Y is a member selected from the group consisting of CH and N;

Z is a member selected from the group consisting of —OR², —NR^{2a}R^{2c}, —C₁₋₆ alkyl-C(O)OR^{2a}, —C(O)R^{2a}, —C(O)OR^{2a}, —OC(O)R^{2a}, —C(O)NR^{2a}R^{2b}, —N(R^{2a})C(O)R^{2b}, —OC(O)NR^{2a}R^{2b}, —N(R^{2a})C(O)OR^{2b}, —NR^{2a}C(O)NR^{2b}R^{2c}, —NR^{2a}S(O)₂R^{2b}, —C₀₋₆ alkyl-S(O)₂NR^{2b}R^{2c},

each R² is independently selected from the group consisting of H, C₁₋₆ alkyl, C₂₋₆ alkenyl, C₂₋₆ alkynyl, —C₁₋₆ alkyl-C(O)OR^{2a},

each R^{2a}, R^{2b} and R^{2c} is independently selected from the group consisting of H, C₁₋₆ alkyl, or alternatively R^{2b} and R^{2c} are combined to form a heterocycloalkyl;

each R³ is independently a member selected from the group consisting of H, C₁₋₆ alkyl and —CH(C(O)O—C₁₋₆ alkyl)₂;

each R⁴ is indpendently a member selected from the group consisting of H, C₁₋₆ alkyl, C₂₋₆ alkenyl, C₂₋₆ alkynyl, C₁₋₆ alkoxy, halogen and C₁₋₆ haloalkyl;

each of subscripts n and o is independently 1 or 2, such that the sum of o and p is 3;

subscript p is from 0 to 4; and

salts, hydrates, solvates, dimers and isomers thereof.

Preferred compounds are described by the formula IA

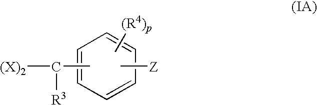

wherein

each X is independently selected from the group consisting of a heteroaryl ring system having from 5 to 10 ring atoms wherein from 1 to 4 ring atoms are heteroatoms each independently selected from the group consisting of N, O and S, wherein at least one of the ring atoms is N, and wherein the heteroaryl ring system is substituted with from 0 to 4 R¹ groups.

Each X is preferably independently selected from the group consisting of:

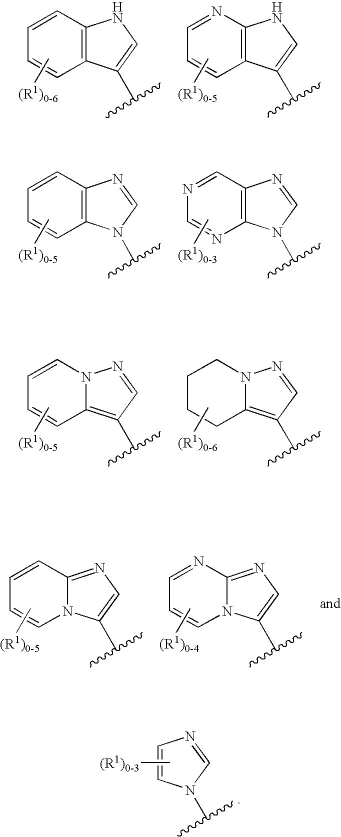

Optionally, each X is the same.

Most preferably each X is:

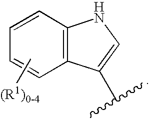

Z is preferably a member selected from the group consisting of —OR², —NR^{2a}R^{2c}, —C(O)OR^{2a}, —NR^{2a}S(O)₂R^{2b} and —C₀₋₆ alkyl-S(O)₂NR^{2b}R^{2c}. More preferably Z is a member selected from the group consisting of —OH, —O—C₁₋₆ alkyl-COOH, —O—C₁₋₆ alkyl, —O—C₂₋₆ alkenyl, —N(—C₁₋₆ alkyl)₂, —NHSO₂CH₃ and

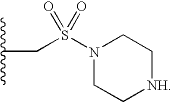

Most preferably Z is —O—C₁₋₆ alkyl-COOH.

Particular preferred compounds include the following:

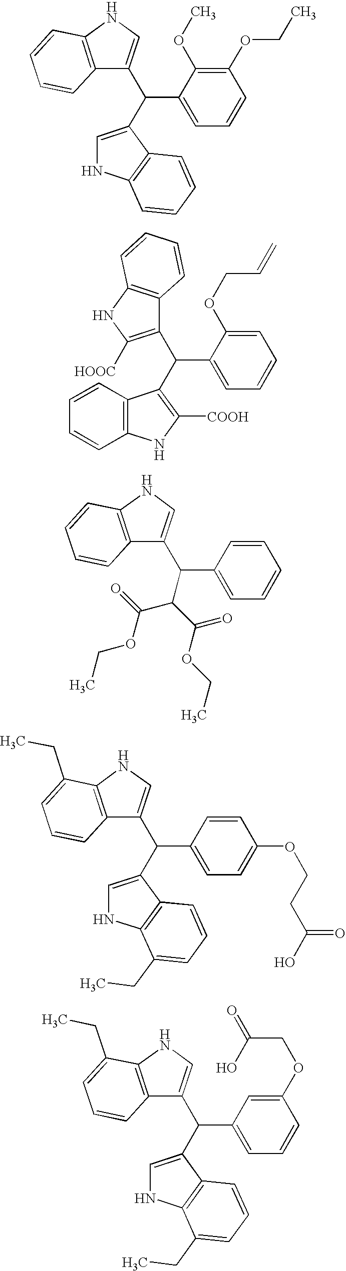

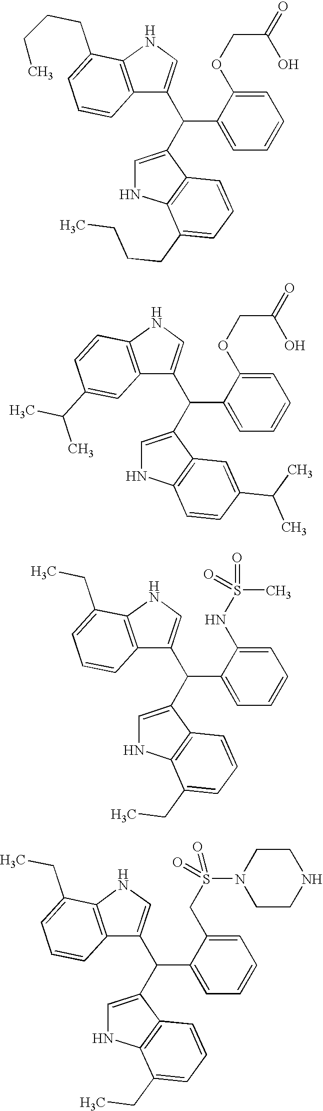

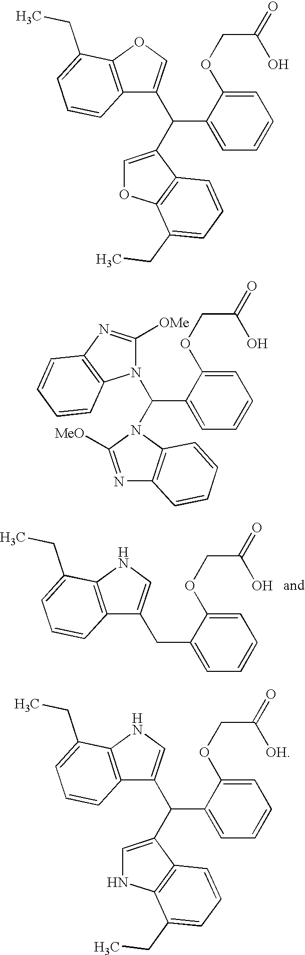

A most preferred compound (7291-0042) has the formula:

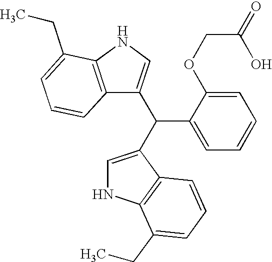

In some compounds, Z includes a carboxylic acid. When Z includes a carboxylic acid, the compounds of the present invention can interact with each other to form a dimer, a complex of two identical molecules linked together through covalent bonds or hydrogen bonding, among others. In the present invention, the dimer forms via hydrogen bonding through the carboxylic acids, such as shown below:

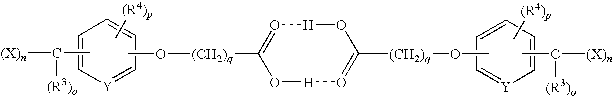

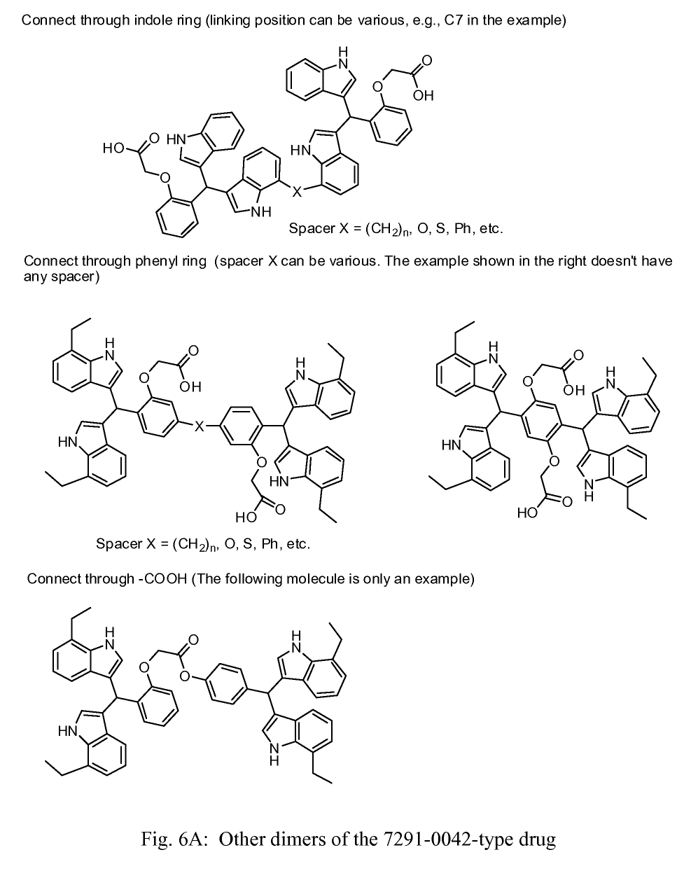

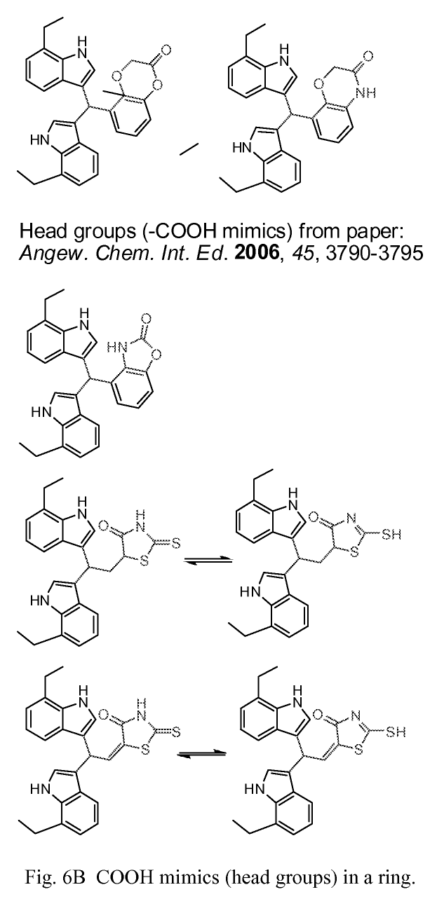

Other functional groups also allow the formation of dimers of the compounds of the present invention. FIG. 6A shows the structure of additional dimers.

Z can also be a group that mimics a carboxylic group. Many such groups also know as head groups have been described in the scientific literature. Some are shown in FIG. 6B. If the main scaffold is kept unchanged many head groups can be used including —O—CH2-COO— n 7291-0042.

In all of the above compound descriptions reference to a compound includes salts, hydrates, solvates, dimers and isomers thereof unless otherwise apparent from the context.

### III. Screening Systems

Screening assays showing activity of compound 7291-0042 are described in the examples. The same and additional screening methods can be used to confirm activity of other compounds. Some screening methods involve contacting under suitable binding conditions (i) a PDZ-domain polypeptide known to bind an oncogenic HPV E6 PL peptide (e.g., MAGI-1), and (ii) the PL peptides in the presence of the test compound. Presence or absence of complex is then detected. The presence of the complex at a level that is statistically significantly higher in the presence of the test compound than in the absence of test compound is an indication that the test compound is an agonist, whereas, the presence of the complex at a level that is statistically significantly lower in the presence of the test compound than in the absence of test compound is an indication that the test compound is an antagonist or inhibitor. Details of such as assays are described by e.g., US Publication No. 2007/0099199. Analogous assays performed for inhibition of binding between a PDZ and PL in cells (see e.g., U.S. Pat. Nos. 5,569,608; 6,297,020; and 6,403,383). An antagonist preferably reduces PDZ-PL binding by at least about 40%, preferably at least about 50%, often at least about 70%, and even as much as at least about 90%.

Compounds can also be tested for activity in inhibiting proliferation of cancerous cells and animal models of cancer. Animal models can be generated by introducing tumor cells into syngeneic mice using standard techniques, e.g. subcutaneous injection, tail vein injection, spleen implantation, intraperitoneal implantation, implantation under the renal capsule, or orthopin implantation, e.g. cervical cancer cells implanted in the cervical tissue. Immunodeficient mice and, in particular, nude mice are particularly useful for such assay (see, e.g., The Nude Mouse in Oncology Research, E. Boven and B. Winograd, eds., CRC Press, Inc. 1991). The cells introduced into such animals can be derived from known cervical cancer cell lines, such as HeLa, SiHa cell lines or cell lines of other cancer. Samples of tumor or cancer cells can be obtained from patients undergoing surgery, using standard conditions, involving freezing and storing in liquid nitrogen.

Transgenic mice studies can be used to examine the activity of compounds on cervical dysplasia and carcinogenesis. Numerous transgenic lines of “high risk” E6-E7 mice under the control of different promoters have been made. Some of these models produced lesions similar to premalignant changes seen in cervical intraepithelial neoplasia (CIN). Some transgenic mice lines express pre-neoplastic proliferating, poorly differentiating epithelial lesions; some do not form neoplastic tumors but form hyperplastic changes in epithelium (Griep et al. 1993, J Virol 67:1373-1384; Greenhalgh et al. 1994, Cell Growth and Differentiation, 1994 5(6):667-75. Transgenic mouse models of other cancer types include mice with knockouts of tumor suppressor genes, and mice having an additional oncogene inserted.

The efficacy of a test compound on a tumor in an animal model can be determined by measuring the size of the tumor before and after treatment with the test compound. The size of implanted tumors can be measured with a slide caliper in two or three dimensions and converted into the corresponding volume by using a mathematical formula.

### IV. Subjects Amenable to Treatment

Subjects amenable to treatment include subjects having a cancer as well as subjects not yet having cancer but at risk of developing a cancer. Examples of cancers treatable by the methods include renal cancer, breast cancer, brain tumors, chronic or acute leukemias including acute myeloid leukemia, chronic myeloid leukemia, acute lymphoblastic leukemia, chronic lymphocytic leukemia, lymphomas {e.g., Hodgkin's and non-Hodgkin's lymphoma, lymphocytic lymphoma, primary CNS lymphoma, T-cell lymphoma) and nasopharangeal carcinomas, melanoma {e.g., metastatic malignant melanoma), prostate cancer, colon cancer, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, Kaposi's sarcoma, epidermoid cancer, squamous cell cancer, environmentally induced cancers including those induced by asbestos, e.g., mesothelioma and combinations of said cancers.

Subjects at risk of developing cancer include subjects with a known genetic risk of cancer as indicated by presence of a genetic variant or relatives having the disease. Subjects at risk also include those have precancerous cells or an infection that predisposes them to cancer. Subjects at risk also include individuals who have or are about to undergo a procedure or experience conveying a risk of developing cancer. Such subjects include those undergoing treatment for a different kind of cancer in that radiotherapy and many forms of chemotherapy are themselves carcinogenic. Such subjects also include those who have been exposed to radiation or carcinogenic chemicals.

The present methods are especially useful for subjects who are infected with an oncogenic strain of HPV or are at risk of such infection. A subject infected with an onocogenic HPV is a subject having cells that contain the oncogenic HPV. Examples of oncogenic strains include 16, 18, 31, 33, and 35. The HPV in the cells may not exhibit any other phenotype (i.e., cells infected with HPV do not have to be cancerous). In other words, cells infected with HPV can be otherwise normal, pre-cancerous (cervical dysplasia) or cancerous cells. Cancers associated with HPV infection include cervical cancer, as well as anal, vulvar, vaginal, penile, and certain types of head and neck cancer.

Cervical cancer is a malignant cancer of the cervix. Almost all subjects having cervical cancer are infected with on oncogenic HPV. However, infection with an oncogenic HPV does not necessarily result in cervical cancer. Cells infected with HPV can be and can remain pre-cancerous. For example, cervical intraepithelial neoplasia, or CIN, is the abnormal growth of precancerous cells in the cervix. Most cases of CIN remain stable, or are eliminated by the subject's immune system without intervention for many years. However a small percentage of cases progress to become cervical cancer. The pathologic types of cervical cancer include (i) cervical intraepithelial neoplasia, the precursor to cervical cancer, (ii) carcinoma malignancies such as squamous cell carcinoma, adenocarcinoma, adenosquamous carcinoma, small cell carcinoma, neuroendocrine carcinoma, and (iii) non-carcinoma malignancies such as melanoma and lymphoma. Cervical pre-cancers and early cancers usually show no symptoms.

Subject at risk of oncogenic HPV infection include subjects having sex at an early age, having many sexual partners, having sex with a partner who has many sexual partners, smokers, those having many children, use birth control pills for a long time, or have HIV infection or Chlamydia infection.

Subjects at a known genetic risk of cervical cancer include those having female relatives who have experienced this disease, and those whose risk is determined by analysis of genetic or biochemical markers. Genetic markers of risk toward cervical cancer can include mutations in the p53 gene. Specifically, the p53 protein with arginine at codon 72 was shown to be more susceptible to E6-induced degradation in vivo than p53 with proline at codon 72 and homozygosity for the allele encoding arginine was found at a significantly higher frequency in the germlines of individuals suffering from HPV-associated squamous carcinoma of the cervix than in the germlines of a control population. Subjects homozygous for the arginine-encoding allele of p53 are seven times more susceptible to HPV-associated cervical tumorigenesis than heterozygotes. (See, Storey et al., 1998, Nature 393:229-234.) Included are also subjects with weak immune systems, who are less able to fight off HPV infection than others.

Subjects presently suffering from cervical cancer can be recognized from cervical cancer testing such as the Papanicolaou (Pap) test, pelvic exam, HPV DNA test, colposcopy, or cervical biopsies, as well as the presence of risk factors described above.

Anal cancer is a type of cancer which arises from the anus, the distal orifice of the gastrointestinal tract. Anal cancer is typically a squamous cell carcinoma that arises near the squamocolumnar junction. Risk factors for anal cancer are similar to those for cervical cancer and, like the cervix, the anal canal contains a transformation zone which can be infected by HPV, leading to the development of lesions. HPV infection is regarded as a likely precursor to anal cancer, rather than as a necessary cause, as it is for cervical cancer (Gagne et al., 2005, J Acquir Immune Defic Syndr. 40:182-189.; Frisch et al., 1999, Cancer Res. 59:753-757). A high prevalence of anal squamous intraepithelial lesions, precursors of anal cancer, have also been reported in sexually active men who have sex with men, which may reflect an ongoing exposure to HPV (Chin-Hong et al, 2005, J Acquir Immune Defic Syndr. 40:463-471).

Penile cancer is a malignant growth found on the skin or in the tissues of the penis, usually originating in the glans and/or foreskin. As with cervical cancer, HPV 16 is commonly found in penile cancers (Salazar et al., 2005, Arch. Androl. 51:327-334). Such subjects include those who have oncogenic HPV infection that can cause penile cancer, those who smoke and those who are not circumcised. The pathology of penile cancer includes (i) pre-cancerous dermatologic lesions, (ii) carcinoma in situ (Bowen disease, Erythroplasia of Queyrat), and (iii) invasive carcinoma of the penis.

Vulvar cancer, a malignant invasive growth in the vulva (external genital organs of the female), accounts for about 4% of all gynecological cancers and typically affects women in later life. HPV16 infection increases the risk of developing vulvar cancers and pre-invasive vulvar lesions by approximately 4.5 times. An association between HPV 18 infection and the development of pre-invasive lesions has also been shown (Bjorge et al., 1997, BMJ 315:646-649). The pathological types of vulvar cancer include squamous cell carcinoma, melanoma, basal cell carcinoma, adenocarcinoma and sarcoma.

Vaginal cancer is a type of cancer that forms in the tissues of the vagina. The vagina leads from the cervix (the opening of the uterus) to the outside of the body. Vaginal cancer types include squamous cell carcinoma and adenocarcinoma. Risk factor include female subjects whose mothers used diethylstilbestrol (DES) to prevent miscarriages or threatened abortions. HPV 16 is shown to be associated with an increased risk of developing vaginal cancers as well as developing pre-invasive vaginal lesions. HPV 18 infection increases the risk of developing pre-invasive lesions (Bjorge et al., 1997, BMJ 315:646-649).

The term “head and neck cancer” refers to a group of biologically similar cancers originating from the upper aerodigestive tract, including the lip, oral cavity (mouth), nasal cavity, paranasal sinuses, pharynx, and larynx. Most head and neck cancers are squamous cell carcinomas, originating from the mucosal lining (epithelium) of these regions. Head and neck cancers often spread to the lymph nodes of the neck, and this is often the first (and sometimes only) manifestation of the disease at the time of diagnosis. Head and neck cancer is strongly associated with certain environmental and lifestyle risk factors, including tobacco smoking, alcohol consumption, and certain strains of HPV.

Some head and neck cancers are more strongly associated with HPV infection than are cancers of other regions of the head and neck. The DNA of HPV has been detected in the tissue of oral and tonsil cancers. Oropharyngeal squamous cell carcinoma are shown to be associated with HPV infection (D'Souza et al., 2007, N. Engl. J. Med. 356:1944-1956). Esophageal cancer is also shown to be associated with HPV infection, notably HPV 16 (Bjorge et al., 1997, Cancer Res. 57:3989-3992).

### V. Methods of Treatment and Pharmaceutical Compositions

The present compounds can be used in methods of treatment or prophylaxis of cancer, such as any of the types described above. In prophylactic application, compounds of the invention are administered to an individual at risk of developing a disease or disorder in a regime (i.e., dose, frequency and route of administration) effective to inhibit or delay or reduce the risk of development of the disease or disorder. For example, for prophylaxis of a cancer, compounds are administered to an individual at risk of developing the cancer in a regime effective to inhibit or delay or reduce the risk of evelopment of the cancer. For prophylaxis of an oncogenic HPV infection, compounds are administered to an individual at risk of HPV infection in a regime effective to inhibit or delay or reduce the risk of HPV infection. In therapeutic applications, compounds are administered to an individual suspected or known to have a disease in a regime effective to reduce, eliminate or inhibit further development of at least one sign or symptom of the disease or its sequellae. For example, for treatment of a cancer, a compound is administered in a regime effective to reduce or eliminate the cancer or at least inhibit further deterioration of the patient's condition due to the cancer. For treatment of an HPV infection, a compound is administered in a regime effective to reduce or eliminate the infection, or at least inhibit further worsening of the infection and its sequellae, such as the development of cervical dysplasia followed by cervical cancer. In both prophylactic and therapeutic regimes, compounds are usually administered in several dosages, e.g., daily, until a sufficient response has been achieved. However, in both prophylactic and therapeutic regimes, the active compounds can be administered in a single dosage. Typically, the treatment is monitored and repeated dosages can be given.

The actual dosage amount of a composition of the present invention administered to a subject can be determined by physical and physiological factors such as body weight, severity of condition, the type of disease being treated, previous or concurrent therapeutic interventions, idiopathy of the subject and on the manner and/or route of administration. A therapeutically effective dose of the present compounds can provide therapeutic benefit without causing substantial toxicity. Toxicity of the compounds can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index (see, e.g., Fingl et al., 1975, In: The Pharmacological Basis of Therapeutics, Ch. 1, p. 1).

Examplary dosages include about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. Examplary ranges In non-limiting examples of a derivable range from the numbers listed herein, a range of about 5 mg/kg/body weight to about 100 mg/kg/body weight, about 5 microgram/kg/body weight to about 500 milligram/kg/body weight, can be administered, based on the numbers described above.

The therapy can be repeated intermittently while symptoms detectable or even when they are not detectable. The therapy can be provided alone or in combination with other drugs effective to treat or effect prophylaxis against HPV infection or cancer, such as chemotherapy, radiation, or surgery.

The compounds of the invention can be administered to a subject alone or in the form of a pharmaceutical composition. Pharmaceutical compositions are preferably in a form suitable for administration to a human including manufacture under GMP practices of the FDA or similar body. Compositions for parenteral administration are preferably substantially isotonic, sterile and substantially free of pyrogens and the like. Pharmaceutical compositions comprising the compounds of the invention can be manufactured by means of conventional mixing, dissolving, granulating, dragee-making, levigating, emulsifying, encapsulating, entrapping or lyophilizing processes. Pharmaceutical compositions can be formulated in conventional manner using one or more physiologically acceptable carriers, diluents, excipients or auxiliaries that facilitate processing of the active peptides or peptide analogues into preparations which can be used pharmaceutically. Proper formulation is dependent on the route of administration chosen.

For topical administration the compounds of the invention can be formulated as solutions, gels, ointments, creams, suspensions and the like.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention can be formulated in aqueous solutions, preferably in physiologically compatible buffers such as Hanks's solution, Ringer's solution, or physiological saline buffer. The solution can contain formulatory agents such as suspending, stabilizing and/or dispersing agents.

Alternatively, the compounds can be in powder form for constitution with a suitable vehicle, e.g., sterile pyrogen-free water, before use.

For transmucosal administration, penetrants appropriate to the barrier to be permeated are used in the formulation. This route of administration can be used to deliver the compounds to the nasal cavity.

For oral administration, the compounds can be readily formulated by combining the active peptides or peptide analogues with pharmaceutically acceptable carriers. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents can be added, such as the cross-linked polyvinylpyrrolidone, agar, or alginic acid or a salt thereof such as sodium alginate.

If desired, solid dosage forms can be sugar-coated or enteric-coated using standard techniques.

For oral liquid preparations such as, for example, suspensions, elixirs and solutions, suitable carriers, excipients or diluents include water, glycols, oils, alcohols. Additionally, flavoring agents, preservatives, coloring agents and the like can be added.

For buccal administration, the compounds can take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can also be formulated in rectal or vaginal compositions such as suppositories or retention enemas, e.g., containing conventional suppository bases such as cocoa butter or other glycerides. Topical compositions and medicated carriers (e.g., medicated “tampon”) can also be used for such routes of administration.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver peptides and peptide analogues of the invention. Certain organic solvents such as dimethylsulfoxide also can be employed, although usually at the cost of greater toxicity. Additionally, the compounds can be delivered using a sustained-release system, such as semipermeable matrices of solid polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

Pharmaceutical composition of the invention can comprise, for example, at least about 0.1% of an active compound. The an active compound can comprise between about 2% to about 75% of the weight of the unit, or between about 25% to about 60%, for example, and any range derivable therein.

D. Kits

Also provided are kits thereof for practicing one or more of the above-described methods. The kits contain a compound of the invention in combination with instructions for using the agent in treatment or infection of an infection by an oncogenic HPV virus.

### EXAMPLES

### Example 1

Identification of Compounds that Disrupt the Interaction Between HPV E6 Protein and MAGI-1 PDZ Domain 1

MAGI-1 PDZ domain 1 was used as a target for rational drug design to identify compounds for the treatment of HPV. The experiment is premised on the finding that the oncogenicity of HPV is based on interaction between an HPV E6 PL and PDZ proteins in vivo. Compounds that specifically disrupt such interaction can provide more effective and less invasive treatments than those currently in use.

Molecular modeling using the structure of MAGI-1 PDZ domain 1 was performed to identify candidate compounds that can disrupt the interaction between HPV E6 protein and MAGI-1 PDZ1. In silico screening with Accelrys software (Accelrys, San Diego, Calif.) was used to model and dock a molecule library (ChemDiv, San Diego, Calif.; Blanca Pharmaceuticals, Mountain View, Calif.) with the PDZ1 domain of the MAGI-1 protein. The molecular modeling was based on finding compounds that had the capability of interacting with MAGI-1 PDZ1 via electrostatic, hydrogen bonding and hydrophobic interactions.

The best hits from in silico screening were subject to screening in a matrix/array competition assay format, i.e., assays where docking of ligands to solid phase PDZ domain in fusion proteins was assessed in the presence and absence of the small molecule competitor as described elsewhere. The compounds were screened for inhibition of the PDZ/PL interactions.

The small molecules were considered as hits based on the OD₄₅₀ readout of the assay. The best of the hits in this analysis were then subject to titration binding studies, i.e., titration of compounds in the same competition assay to estimate the IC₅₀ value. The results of these screens are shown in Table 1.

TABLE 1

| | Magi1 | PSD95 | PSD95 | PSD95 | | |
|---|---|---|---|---|---|---|
| Molecule | d1 | d1 | d2 | d3 | Shank1 | Tip1 |
| D008-0168 | >250 | 159.36 | 176.5 | >250 | 212.2 | >250 |
| 2357-3224 | 160.11 | >250 | >250 | 210.23 | 211.35 | >250 |
| E544-0129 | 60.76 | 2.5 | 4.98 | 3.47 | 9.07 | >250 |
| 0620-0057 | 236.97 | 2.7 | 14.88 | 8.194 | >250 | >250 |
| 7291-0042 | 86.89 | >250 | >250 | >250 | >250 | >250 |
| 3289-2331 | 130.33 | >250 | >250 | >250 | >250 | >250 |
| 1193-0076 | >250 | >250 | >250 | >250 | >250 | >250 |
| 3807-2058 | >250 | >250 | >250 | >250 | >250 | >250 |
| 2817-0095 | 86.43 | 183.35 | >250 | 99.91 | >250 | >250 |
| C450-0454 | >250 | 206.07 | >250 | >250 | >250 | >250 |
| 3558-0042 | >250 | >250 | >250 | >250 | >250 | >250 |

Based on the in silico screening, various compounds were identified to disrupt PDZ/PL interactions. As shown on Table 1, compound 7291-0042 disrupted the interaction between HPV E6 protein and MAGI-1 PDZ1 with greatest specificity.

### Example 2

### Effect of Compound 7291-0042 on Cell Migration

The effect of compound 7291-0042 on the rate of cell migration and proliferation was determined on three different cell lines, 3Y1 pBlast, 3Y1 pBlast-E6-16 and 3Y1 pBlast E6-16 ΔPL.

Plasmid constructs of HPV E6-16 (wild type) and HPV E6-16 ΔPL were generated using the vector pBlast (InvivoGen, Toulouse, France). Recombinant plasmids were generated by recombinant DNA cloning methods known in the art.

These constructs were transfected into rat 3Y1 primary cells using the LipofectAMINE™ 2000 Reagent (Invitrogen Cat#11668-027) and accompanying protocol. pBlast without insert was transfected as a negative control. Cells were incubated at 37° in RPMI media with non-essential amino acids, 10% FBS, and 1 ug/mL G418 until confluent (about 4 days).

Each of the three transfected cell lines were seeded onto a 96-well filter plate with a feeder tray and lid in RPMI media with a gradient of 0.5% to 10% FBS and non-essential amino acids. Three different treatments were applied to the cell lines. The cells were treated with either 40 μM or 60 μM of the compound 7291-0042, or DMSO. Each filter insert had a polycarbonate membrane with 8 μm pores. The undersides of the filters were left uncoated. Invasive and migratory cells migrated through the pores of the filter in response to a chemoattractant and clinged to the bottom of the polycarbonate membrane. Cell migration through the membrane was quantified by staining the cells that attach to the lower side of the membrane with a colorometric dye at OD₅₆₀ after 24 hours with a microplate reader.

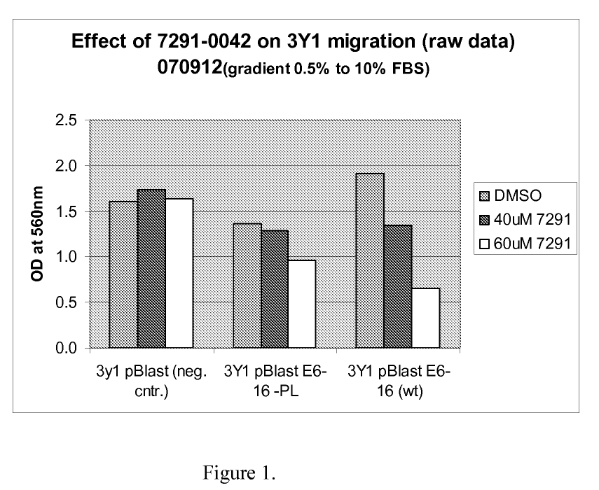

The results of migration assays showed that treatment of compound 7291-0042 restored baseline cell migration. In other words, 3Y1 pBlast-E6-16 cells which express wild type E6 protein showed enhanced cell migration (See FIG. 1, DMSO control). But, when these cells were treated with 40 μM of compound 7291-0042, the rate of cell migration was similar to that 3Y1 pBlast E6-16 ΔPL, cells that lack the E6 PL motif. See FIG. 1. Cell migration of HPV E6-16 cells has been shown to be PL dependent as demonstrated by E6 wild type cells migrating faster than ΔPL cells. The results showed that compound 7291-0042 disrupted the ability of the PL motif to bind to the PDZ domain and thereby restored the rate of cell migration to that of the ΔPL cells.

### Example 3

### Effect of Compound 7291-0042 on Cell Proliferation

Compound 7291-0042 was further analyzed for its ability to block the development of other cancerous characteristics in cells by a cell proliferation assay. The three cell lines described in the Example 2 were seeded onto a 12-well plate, and allowed to adhere and grow to confluent (about 24 hours) in EMEM media with 10% FBS and non-essential amino acids. 100 μl of compound 7291-0042 at concentrations 0 μM, 20 μM, 40 μM, and 80 μM was applied to the cells. The cells were measured at 24, 48, 72 and 96 hours after addition of the compound.

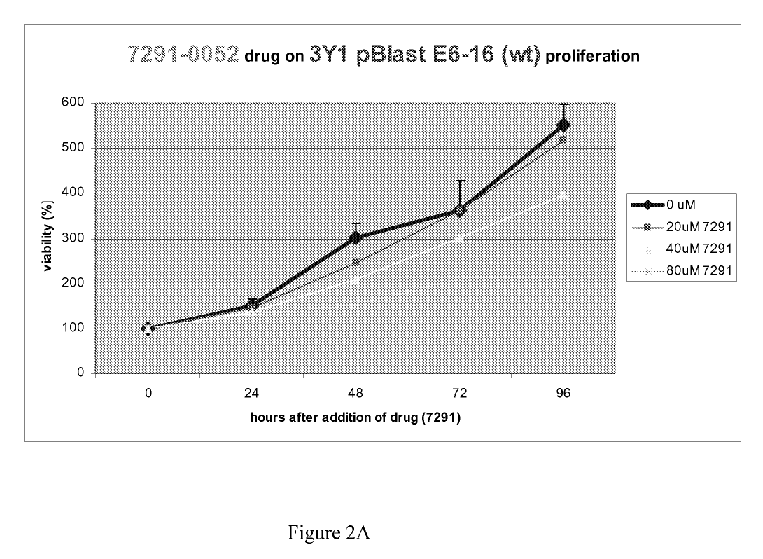

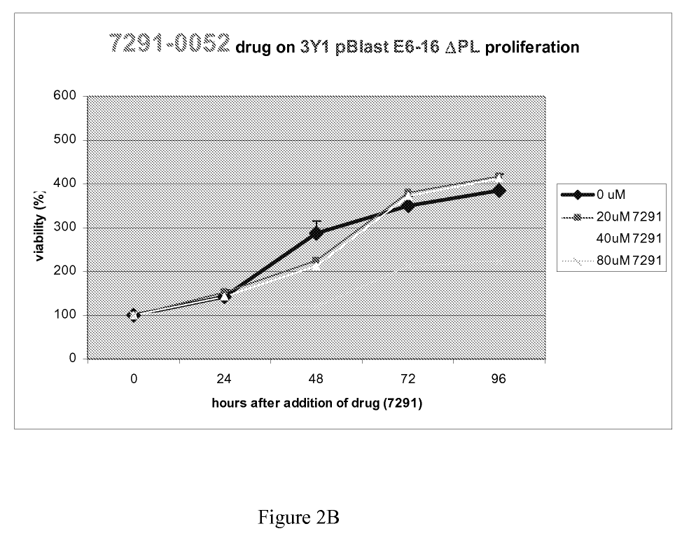

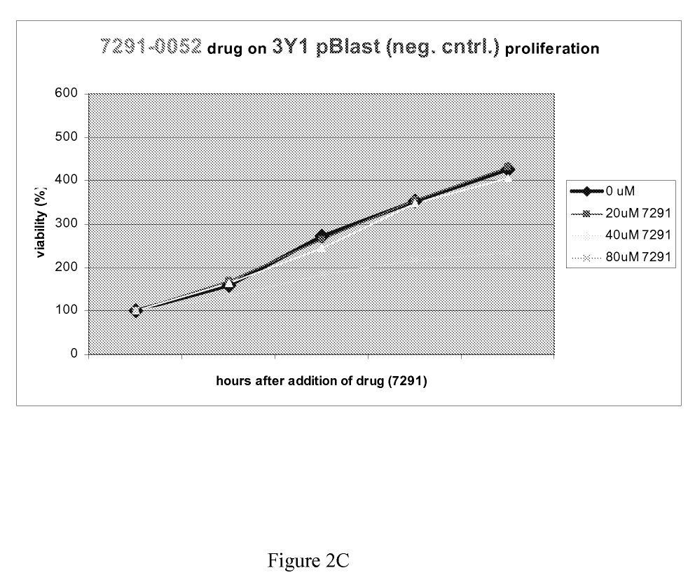

The results of cell proliferation assays showed that treatment of compound 7291-0042 restored baseline rate of cell proliferation. The 3Y1 pBlast-E6-16 cells which express wild type E6 protein showed enhanced cell proliferation without addition of compound 7291-0042. But, when these cells were treated with 40 μM of compound 7291-0042, the rate of cell proliferation was similar to that 3Y1 pBlast E6-16 ΔPL and 3Y1 pBlast. See FIGS. 2A, 2B and 2C.

Therefore, based on these results in Examples 2 and 3, the PL motif on E6 proteins from oncogenic strains of HPV is essential for the development of cancerous characteristics such as cell migration and cell proliferation. The results demonstrated that the compound 7291-0042 was able to disrupt the binding of E6 PL motif with PDZ domain and thereby block the development of such cancerous characteristics in cells.

### Example 4

### Drug Toxicity Studies of Compound 7291-0042 in Cells

To determine the cytotoxicity of the compound 7291-0042, drug toxicity studies were performed in HPV-positive (E6 transformed) cervical cancer cell lines SiHa and HeLa and HPV-negative cervical cancer cell line C33a. C33a and SiHa cells were seeded onto a 12-well plate, allowed to adhere and grown to 10,000 cells per well. HeLa cells were seeded, allowed to adhere and grown to 5,000 cells per well. 100 μl of compound 7291-0042 at a concentration of 60 μM or DMSO was added to the cells 24 hours after plating. Cytotoxicity of the cells was assessed by the WST-1 calorimetric assay. In brief, the calorimetric assay is based on the cleavage of the tetrazolium salt WST-1 to a formazan-class dye by mitochondrial succinate-tetrazolium reductase in viable cells. As the cells proliferate, more WST-1 is converted to the formazan product. The quantity of formazan dye is directly related to the number of metabolically active cells, and can be quantified by measuring the absorbance at 420-480 nm (Aₘₐₓ 450 nm) in a multiwell plate reader.

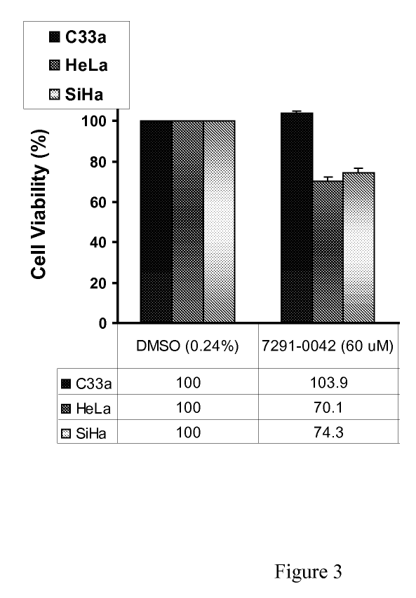

The results showed that when treated with compound 7291-0042, E6 transformed cervical cancer cell lines SiHa and HeLa cells were selectively killed, whereas HPV-negative C33a cells remained viable. In the DMSO control, all cells remained viable. See FIG. 3. The results indicated that the compound 7291-0042 has a selective toxicity for E6 cervical cancer cells, providing evidence that compound 7291-0042 can be useful as a drug in treating cervical cancer.

### Example 5

### Effect of Compound 7291-0042 on E6 Oncoprotein Expression

To determine whether the compound 7291-0042 has an effect on E6 protein expression, cervical cancer cell line HeLa was used. HeLa cells were seeded, allowed to adhere and grown to 0.5×10⁶ cells per well. Compound 7291-0042 at a concentration of 60 μM, 80 μM, 100 μM, 120 μM, or DMSO was added to the cells 24 hours after plating and incubated for 24 hours. Cells that were untreated served as an additional control. Western blot was performed to determine the relative amounts of the E6 protein present in the different samples. Briefly, the cells were homogenized in a lysate buffer and the protein samples were separated using SDS-PAGE and transferred to a membrane for detection. The membrane was incubated with an anti-HPV18-E6 antibody, labeled with a secondary antibody-enzyme conjugate and detected. To ensure that the amount of protein loaded for each sample was similar, the membrane was stripped and labeled with an antibody that recognizes a protein encoding a housekeeping gene. For this experiment, an antibody against glyceraldehyde-3-phosphate dehydrogenase (GAPDH) was used.

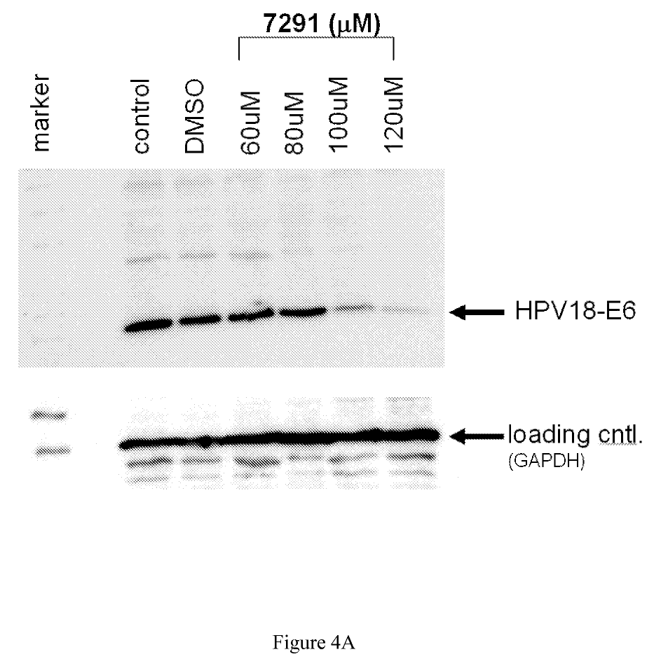

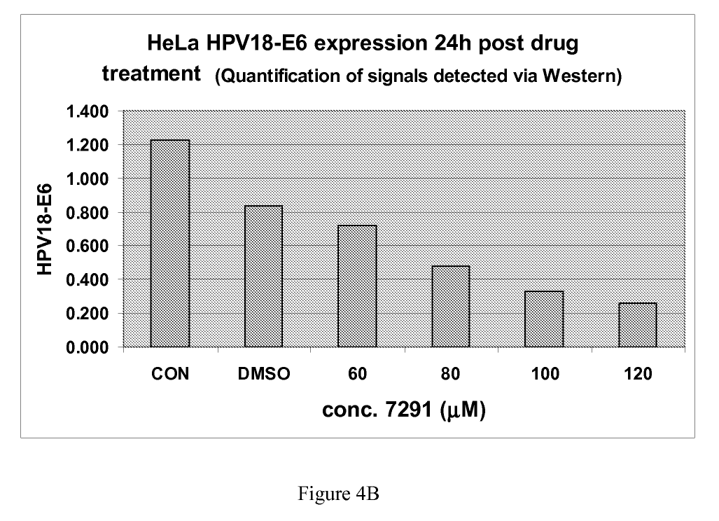

The signals detected in the Western blot were quantified. The results showed that when HeLa cells were treated with compound 7291-0042, the amount of E6 onco protein was decreased compared to those of the DMSO control and the untreated cells (see FIGS. 4A and 4B).

### Example 6

### Effect of Compound 7291-0042 on p53 Tumor Suppressor Protein Expression

To determine whether the compound 7291-0042 has an effect on p53 protein expression, cervical cancer cell line HeLa was used. HeLa cells were seeded, allowed to adhere and grown to 0.5×10⁶ cells per well. 1 ml of compound 7291-0042 at a concentration of 80 μM, 100 μM, 120 μM, or DMSO was added to the cells 24 hours after plating and incubated for 24 hours. Immunocytochemistry with anti-p53 monoclonal antibody was performed to determine the relative amounts of the p53 protein present in the different cell samples.

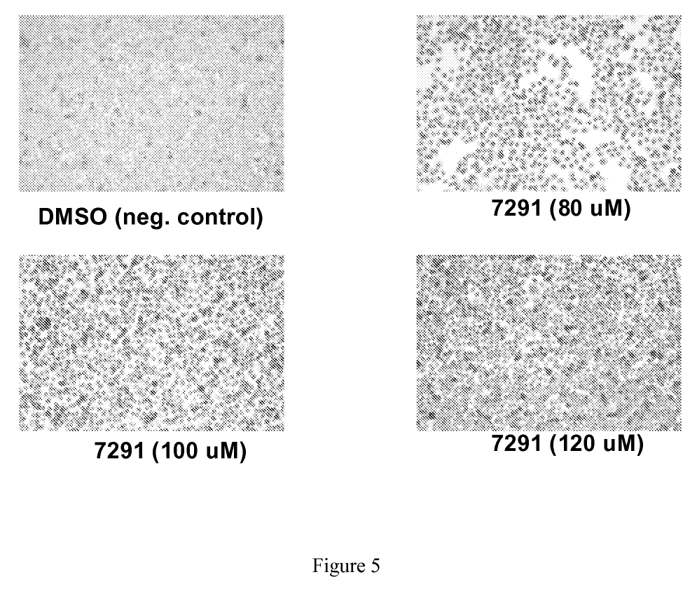

The results showed that when HeLa cells were treated with compound 7291-0042, the amount of p53 tumor suppressor protein was increased compared to that of the DMSO control cells. (see FIG. 5). The cytology of the cells after treatment of the compound shows morphological changes associated with cell shrinking.

### Example 7

Compound 7291-0042 Selectively Induces Apoptosis in HPV-Positive Cervical Cancer Cells

To determine whether the compound 7291-0042 induces apoptosis in HPV-positive cervical cancer cell lines, HeLa cells and the HPV-negative cell line C33A were used. HeLa and C33A cells were seeded at 1-2×10⁶ in 10-cm dishes and allowed to adhere and grow to 80-90% confluency. Compound 7291-0042 at a concentration of 60 μM, 80 μM, 100 μM, 120 μM, or DMSO was added to the cells 24 hours after plating and incubated for 48 hours. TUNEL assay was performed for the specific quantitation of apoptotic cells within a cell population using the DeadEnd Fluorometric TUNEL System from Promega (Madison, Wis.). Briefly, the cells were harvested by trypsinization and washed in PBS. Cells were then fixed in 4% formalin and incubated in buffer containing nucleotides and rTdT enzyme for 1 hour at 37° C. After terminating the reaction by adding EDTA, cells were washed with PBS and permeabilized with Triton X-100 and propidium iodide was added to stain all the cells. Cells were then analyzed by flow cytometery. Apoptosis signal was quantified by measuring green fluorescence of fluorescein-12-dUTP at 520±20 nm and red fluorescence of propidium iodide at >620 nm.

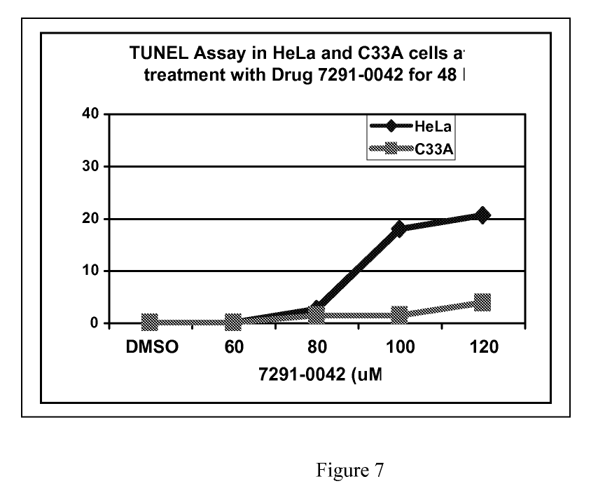

As shown in FIG. 7, 7291-0042 induced apoptosis in 20% of the HeLa cells at a 120 M while only 4% of the C33A cells were apoptotic at this concentration. This result documents that 7291-0042 selectively induces apoptosis in HPV-positive cancer cells.

Although the foregoing invention has been described in detail for purposes of clarity of understanding, it will be obvious that certain modifications may be practiced within the scope of the appended claims. All publications and patent documents cited herein are hereby incorporated by reference in their entirety for all purposes to the same extent as if each were so individually denoted. Unless otherwise apparent from the context, any embodiment, element, feature, or step of the invention can be used in combination with any other. From the foregoing it will be apparent that the invention provides for a number of uses. For example, the invention provides for the use of any of the compounds described above in the treatment, prophylaxis or diagnosis of disease, particularly cancer and/or HPV infection and its sequellae.

1. A method of treating or effecting prophylaxis against an infection by an oncogenic human papilloma virus (HPV), comprising administering to a subject having or at risk of HPV infection an effective regime of a compound that treats or effects prophylaxis of the infection or its sequellae, and has a formula I: 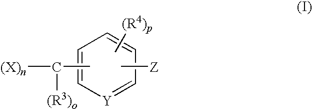
wherein
each X is a heteroaryl ring system having from 5 to 10 ring atoms wherein from 1 to 4 ring atoms are heteroatoms each independently selected from the group consisting of N, O and S, wherein the heteroaryl ring system is substituted with from 0 to 6 R¹ groups;
each R¹ is indpendently selected from the group consisting of H, C₁₋₆ alkyl, C₂₋₆ alkenyl, C₂₋₆ alkynyl, C₁₋₆ alkoxy, halogen, C₁₋₆ haloalkyl, —C₀₋₆ alkyl-OR^{1a}, —NR^{1a}R^{1b}, —CN, —C(O)R^{1a}, —C(O)OR^{1a}, —OC(O)R^{1a}, —C(O)NR^{1a}R^{1b}, —N(R^{1a})C(O)R^{1b}, —OC(O)NR^{1a}R^{1b}, —N(R^{1a})C(O)OR^{1b}, —NR^{1a}C(O)NR^{1b}R^{1c}, —NO₂, —C₀₋₆ alkyl-aryl, heteroaryl, cycloalkyl and heterocycloalkyl;
each of R^{1a}, R^{1b} and R^{1c} are independently selected from the group consisting of H and C₁₋₆ alkyl;
Y is a member selected from the group consisting of CH and N;
Z is a member selected from the group consisting of —OR², —NR^{2a}R^{2c}, —C₁₋₆ alkyl-C(O)OR^{2a}, —C(O)R^{2a}, —C(O)OR^{2a}, —OC(O)R^{2a}, —C(O)NR^{2a}R^{2b}, —N(R^{2a})C(O)R^{2b}, —OC(O)NR^{2a}R^{2b}, —N(R^{2a})C(O)OR^{2b}, —NR^{2a}C(O)NR^{2b}R^{2c}, —NR^{2a}S(O)₂R^{2b}, —C₀₋₆ alkyl-S(O)₂NR^{2b}R^{2c},
each R² is independently selected from the group consisting of H, C₁₋₆ alkyl, C₂₋₆ alkenyl, C₂₋₆ alkynyl, —C₁₋₆ alkyl-C(O)OR^{2a},
each R^{2a}, R^{2b} and R^{2c} are indpendently selected from the group consisting of H, C₁₋₆ alkyl;
alternatively R^{2b} and R^{2c} are combined to form a heterocycloalkyl;
each R³ is independently a member selected from the group consisting of H, C₁₋₆ alkyl and —CH(C(O)O—C₁₋₆ alkyl)₂;
each R⁴ is indpendently a member selected from the group consisting of H, C₁₋₆ alkyl, C₂₋₆ alkenyl, C₂₋₆ alkynyl, C₁₋₆ alkoxy, halogen and C₁₋₆ haloalkyl;
each of subscripts n and o are independently 1 or 2, such that the sum of o and p is 3;
subscript p is from 0 to 4; and
salts, hydrates, solvates, dimers and isomers thereof.

2. The method of claim 1, wherein the compound has a formula IA 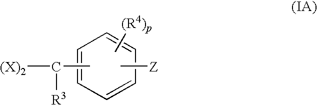
wherein
each X is independently selected from the group consisting of a heteroaryl ring system having from 5 to 10 ring atoms wherein from 1 to 4 ring atoms are heteroatoms each independently selected from the group consisting of N, O and S, wherein at least one of the ring atoms is N, and wherein the heteroaryl ring system is substituted with from 0 to 4 R¹ groups.

3. The method of claim 2, wherein each X is independently selected from the group consisting of:

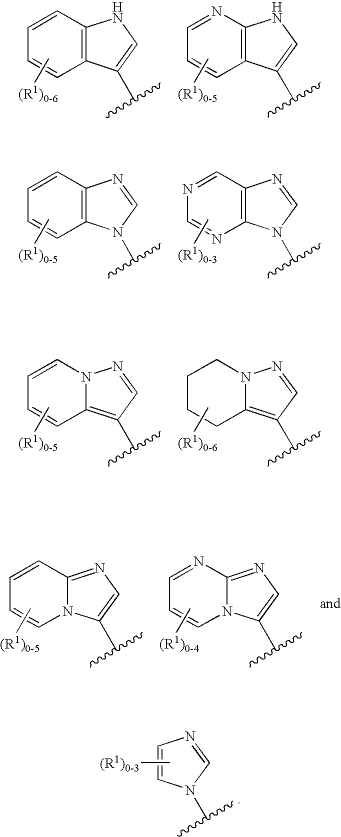

4. The method of claim 3, wherein each X is the same.

5. The method of claim 3, wherein each X is:

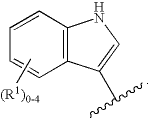

6. The method of claim 1, wherein Z is a member selected from the group consisting of —OR², —NR^{2a}R^{2c}, —C(O)OR^{2a}, —NR^{2a}S(O)₂R^{2b} and —C₀₋₆ alkyl-S(O)₂NR^{2b}R^{2c}.

7. The method of claim 6, wherein Z is a member selected from the group consisting of —OH, —O—C₁₋₆ alkyl-COOH, —O—C₁₋₆ alkyl, —O—C₂₋₆ alkenyl, —N(—C₁₋₆ alkyl)₂, —NHSO₂CH₃ and

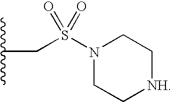

8. The method of claim 7, wherein Z is —O—C₁₋₆ alkyl-COOH.

9. The method of claim 1, wherein the compound is selected from the group consisting of:

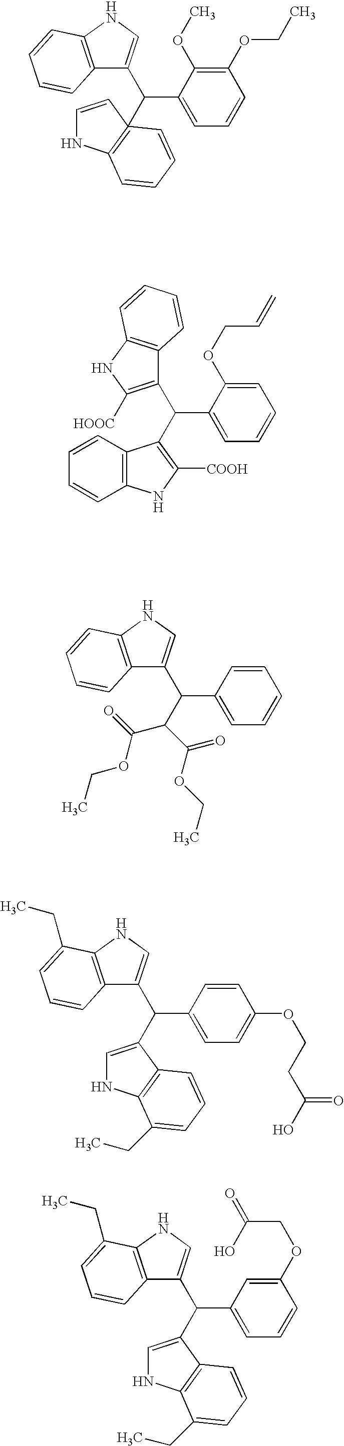

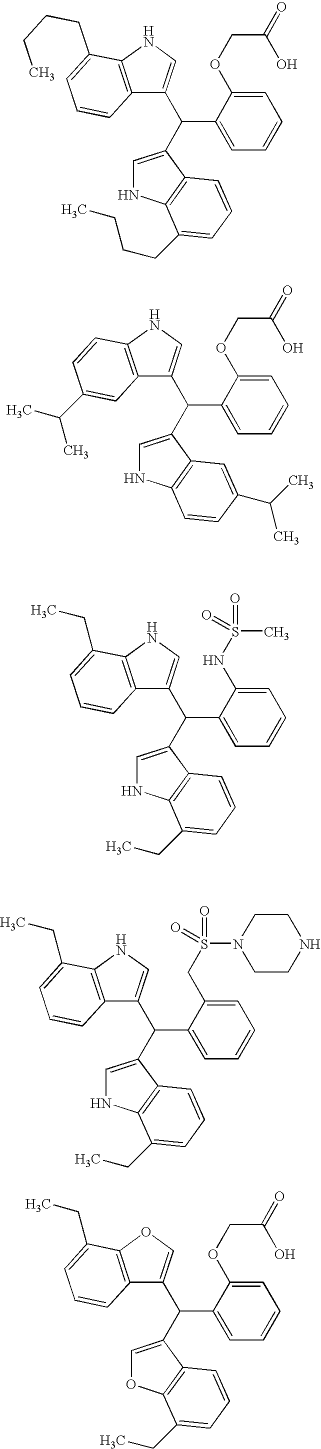

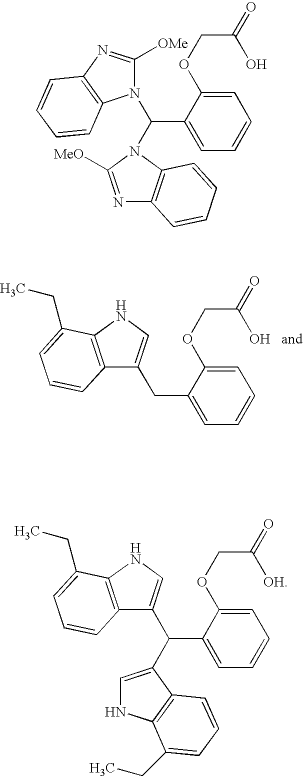

10. The method of claim 1, wherein the compound has the formula:

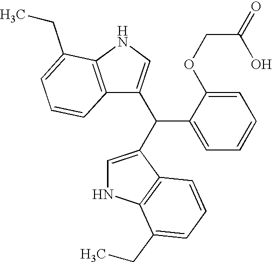

11. The method of claim 1 wherein the compound inhibits binding of HPV E6 protein to a polypeptide comprising the amino acid sequence of a first PDZ domain from MAGI-1.

12. The method of claim 1, wherein the subject is infected with HPV.

13. The method of claim 1, wherein the subject has cervical cancer.

14. The method of claim 1, wherein the subject has cervical dysplasia.

15. The method of claim 1, wherein the subject is at risk of HPV infection.

16. The method of claim 1, comprising administering to a subject having or at risk of cancer an effective regime of the compound, whereby the compound treats or effects prophylaxis of the cancer.

17. The method of claim 16, wherein the subject is infected with an oncogenic human papilloma virus.

18. The method of claim 16, wherein the cancer is cervical cancer, vaginal cancer, anal cancer or head and neck cancer.

19. The method of claim 16, wherein the cancer is breast cancer, ovarian cancer, brain cancer, leukemia or lymphoma.

20. The method of claim 16, wherein the cancer is cervical cancer.

21. A compound of formula I in claim 1.

22. The compound of claim 21 for use in treating or effecting prophylaxis of cancer.

23. The compound of claim 21 for use in treating or effecting prophylaxis of HPV infection.

24-25. (canceled)
